(19) United States
(12) Patent Application Publication
(10) Pub. No.: US 2009/0318526 A1
(43) Pub. Date: Dec. 24, 2009

(54) Use of cannabinoid receptor agonists as hypothermia inducing drugs for the treatment of ischemia

(76) Inventors: Uno Jacob Weber, Bagsvaerd (DK); Jacob Gotfredsen, Copenhagen (DK)

Correspondence Address: NIXON & VANDERHYE, PC, 901 NORTH GLEBE ROAD, 11TH FLOOR, ARLINGTON, VA, 22203, US

(21) Appl. No.: 12/308,097

(22) PCT Filed: Jun. 8, 2007
(86) PCT No.: PCT/DK2007/000279

§ 371 Date: Apr. 1, 2009

(30) Foreign Application Priority Data
Jun. 8, 2006 (DK) PA 2006 00777
Mar. 6, 2007 (DK) PA 2007 00337

(51) Int. Cl. A61K 31/405; C07D 209/04

(52) U.S. Cl. 514/415; 548/469

(57) ABSTRACT

The present invention relates to the use of a cannabinoid receptor agonist for use in induction of hypothermia in a human being for the prophylaxis and treatment of ischemia.

### FIELD OF INVENTION

The present invention relates to the use of compounds for the induction of hypothermia for the prophylaxis and treatment of ischemia. lschemia is the lack of oxygenated blood flow to various body parts and may result from apoplexia, cardiac arrest and asphyxia.

### BACKGROUND OF INVENTION

Ischemia is the lack of oxygenated blood flow to various body parts and organs. Cerebral ischemia is an ischemic condition where the brain or parts of the brain do not receive enough blood flow to maintain normal neurological function. Cerebral ischemia can be the result of various serious diseases such as stroke and cardiac arrest, or the result of arterial obstruction such as strangulation. Severe or prolonged cerebral ischemia will result in unconsciousness, brain damage or death.

The neuroprotective efficacy of induced hypothermia following or during ischemia of the brain is evident in experimental animal models of stroke [1-11]. In humans, two trials conducted in cardiac arrest patients have shown improved neurological outcome of inducing hypothermia [12;13]. The therapeutic hypothermia did not increase the complication rate in these two trials and the use of induced hypothermia in comatose survivors of cardiac arrest is now recommended internationally [14].

Hypothermia counteracts ischemic brain damage by several mechanisms:
1. Ischemia induces opening of the blood-brain barrier, a process that seems to be very sensitive to brain temperature [15]. This is evident from studies of tracers and their migration across the blood-brain barrier, in which hypothermia attenuates extravasation several hours after ischemia [16] and prevents vasogenic oedema [17].
2. Reperfusion after brain ischemia results in the production of free radicals, which causes peroxidation and destruction of membrane lipids [18]. Hypothermia prevents the production of free radicals such as hydroxyl and nitric oxide during reperfusion after brain ischemia [19;20].
3. Amino acids, such as glutamate, aspartate, and glycine, act as excitotoxic neurotransmitters by over stimulation of neurons in the vicinity of ischemic damage, which causes further injury. Hypothermia lowers the release and may even cause a more rapid reuptake of these transmitters [21-24]. Release of excitotoxic neurotransmitters might also cause progressive neuronal death in the penumbra in stroke patients [22], and hypothermia after cerebral ischemia could attenuate this process.
4. During ischemia, cellular metabolism in the penumbra undergoes significant changes. As the neurons continue to fire, potassium ions flood into the extracellular space, calcium ions flow into the neurons leading to cytoskeletal degradation, and ATP concentrations fall as energy depletion continues [25]. Hypothermia reduces calcium influx and the subsequent breakdown of intracellular structures [26], improves potassium ion homoeostasis [27], and helps metabolic functions such as calcium or calmodulin-dependent protein kinase activity to recover [28;29].
5. By lowering of neutrophil and microglial activation after ischemia, hypothermia also has an anti-inflammatory effect [30;31].
6. Apoptosis and DNA changes are crucial stages in delayed neuronal death after transient cerebral ischemia [32]. Hypothermia directly inhibits apoptosis and may also increase endogenous production of the anti-apoptotic protein Bcl-2 [34]. Hypothermia may even have effects at the DNA level: A slight lowering of brain temperature results in less DNA fragmentation [35] and less apoptosis [36].

Induction of hypothermia by lowering of the core temperature of the body has been attempted by mechanical cooling devices such as surface cooling and cooling using catheters placed in a large vessel. However, these mechanical inducers of hypothermia have been shown to have considerable unwanted side effects. These side effects include shivering, serious infections and lung puncture. Shivering causes an increased exertion of the heart of the patient, and this will in some cases result in ischemia of the heart and thereby increased morbidity and mortality.

The regulation of the core temperature of the body by a pharmaceutical composition comprising a compound capable of inducing hypothermia would not only solve the problem of preventing the effects of ischemia, but also be relevant as a safer and less expensive alternative to the currently employed mechanical methods.

Leker et al. [37] described that the cannabinoid HU-210 did provide hypothermia and protection against ischemic damages in Sprague-Dawley rats. However, Leker et al. observed hemodynamic and behavioural side effects due to the treatment in the rats, such side effects precluding the drug used in humans.

The results found by Leker et al. cannot be translated into treatment of humans due to a variety of reasons. Leker et al. themselves do state that the cannabinoid of their choice provides too serious side effects to be used for human treatment. Furthermore, according to Howlett et al. [42] with respect to cannabinoid receptors choice it is not possible to translate rat or monkey results to human results, since cannabinoid receptors are expressed differently in the different species accounting for the different effects seen when administering the same cannabinoid to various species. Herkenham et al. [43] also demonstrate that cannabinoid effects in humans and dogs differ.

Another reason for the variation observed may be the less than 100 % homology in cannabinoid receptors of the various species.

Furthermore, medical induction of hypothermia in animals having a body weight of 300 g differs significantly from induction of hypothermia in humans having an average body weight of 70-75 kg, ie. a 250-fold difference in body weight and thus volume that has to be reduced in temperature when inducing hypothermia.

Cannabinoid-containing drugs have been administered to humans for the treatment of pain, however no reports of significant hypothermia observed in humans during such treatment has been described.

### SUMMARY OF INVENTION

The present inventors have found that it is possible to medically induce hypothermia in human beings by administration of a cannabinoid. Thus, the present invention relates to the induction of hypothermia in humans in a predictable and dose responsive fashion by use of a pharmaceutical composition comprising a compound capable of inducing hypothermia, thereby benefiting patients suffering from illnesses characterized by tissue ischemia and anoxia. The inventors have found that such hypothermic effects can be obtained in humans as a result of compounds such as cannabinoids or cannabimimetic agonists reaching and binding to cannabinoid receptors.

Thus the present invention discloses the use of a compound for the induction of hypothermia for the preparation of a medicament for the treatment of ischemia in an individual.

It is also an aspect of the present invention to provide a medicament comprising a compound capable of inducing hypothermia in an individual.

A kit of parts comprising the medicament as herein disclosed is yet an aspect of the present invention.

Furthermore, the use of a compound according for the preparation of a medicament for obviating the induction of hypothermia in an individual, is an aspect of the present invention.

### DETAILED DESCRIPTION OF THE INVENTION

### Definitions

Agonist: A cannabinoid receptor agonist is a cannabinoid or a cannabimimetic compound.

Antagonist: A cannabinoid receptor antagonist is a substance capable of inhibiting the effect of a cannabinoid receptor agonist.

Alcohol: A class of organic compounds containing one or more hydroxyl groups (OH). In this context a saturated or unsaturated, branched or unbranched hydrocarbon group sitting as a substituent on a larger molecule.

Alicyclic group: the term “alicyclic group” means a cyclic hydrocarbon group having properties resembling those of aliphatic groups.

Aliphatic group: in the context of the present invention, the term “aliphatic group” means a saturated or unsaturated linear or branched hydrocarbon group. This term is used to encompass alkyl, alkenyl, and alkynyl groups, for example.

Alkyl group: the term “alkyl group” means a saturated linear or branched hydrocarbon group including, for example, methyl, ethyl, isopropyl, t-butyl, heptyl, dodecyl, octadecyl, amyl, 2-ethythexyl, and the like.

Alkenyl group: the term “alkenyl group” means an unsaturated, linear or branched hydrocarbon group with one or more carbon-carbon double bonds, such as a vinyl group.

Alkynyl group: the term “alkynyl group” means an unsaturated, linear or branched hydrocarbon group with one or more carbon-carbon triple bonds.

Amphiphil: substance containing both polar, water-soluble and nonpolar, water-insoluble groups.

Aromatic group: the term “aromatic group” or “aryl group” means a mono- or polycyclic aromatic hydrocarbon group.

Cannabinoid: Compound capable of binding to a cannabinoid receptor and isolated from or identical to a compound isolated from an organism such a plant or animal. In the present context any compound capable of binding a cannabinoid receptor.

Cannabimimetic: Compound capable of binding to a cannabinoid receptor and produced or synthesized chemically by standard techniques known in the art. In the present context any compound capable of binding a cannabinoid receptor.

Cyclic group: the term “cyclic group” means a closed ring hydrocarbon group that is classified as an alicyclic group, aromatic group, or heterocyclic group.

Cycloalkenyl: means a monovalent unsaturated carbocyclic radical consisting of one, two or three rings, of three to eight carbons per ring, which can optionally be substituted with one or two substituents selected from the group consisting of hydroxy, cyano, lower alkenyl, lower alkoxy, lower haloalkoxy, alkenylthio, halo, haloalkenyl, hydroxyalkenyl, nitro, alkoxycarbonenyl, amino, alkenylamino, alkenylsulfonyl, arylsulfonyl, alkenylaminosulfonyl, arylaminosulfonyl, alkylsulfonylamino, arylsulfonylamino, alkenylaminocarbonyl, arylaminocarbonyl, alkenylcarbonylamino and arylcarbonylamino.

Cycloalkyl: means a monovalent saturated carbocyclic radical consisting of one, two or three rings, of three to eight carbons per ring, which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino and arylcarbonylamino.

Cationic group: A chemical group capable of functioning as a proton donor when a compound comprising the chemical group is dissolved in a solvent, preferably when dissolved in water.

Form a ring: means that the atoms mentioned are connected through a bond when the ring structure is formed.

Group: (Moiety/substitution) as is well understood in this technical area, a large degree of substitution is not only tolerated, but is often advisable. Substitution is anticipated on the materials of the present invention. As a means of simplifying the discussion and recitation of certain terminology used throughout this application, the terms “group” and “moiety” are used to differentiate between chemical species that allow for substitution or that may be substituted and those that do not allow or may not be so substituted. Thus, when the term “group” is used to describe a chemical substituent, the described chemical material includes the unsubstituted group and that group with O, N, or S atoms, for example, in the chain as well as carbonyl groups or other conventional substitution. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, t-butyl, and the like. The same definitions apply to “alkenyl group” and “alkenyl moiety”; to “alkynyl group” and “alkynyl moiety”; to “cyclic group” and “cyclic moiety; to “alicyclic group” and “alicyclic moiety”; to “aromatic group” or “aryl group” and to “aromatic moiety” or “aryl moiety”; as well as to “heterocyclic group” and “heterocyclic moiety”.

Heterocyclic group: the term “heterocyclic group” means a closed ring hydrocarbon in which one or more of the atoms in the ring is an element other than carbon (e.g., nitrogen, oxygen, sulphur, etc.).

Heterocyclyl means a monovalent saturated cyclic radical, consisting of one to two rings, of three to eight atoms per ring, incorporating one or two ring heteroatoms (chosen from N, O or S(O)₀₋₂, and which can optionally be substituted with one or two substituents selected from the group consisting of hydroxyl, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminofarbonyl, arylaminocarbonyl, alkylcarbonylamino, or arylcarbonylamino.

Heteroaryl means a monovalent aromatic cyclic radical having one to three rings, of four to eight atoms per ring, incorporating one or two heteroatoms (chosen from nitrogen, oxygen, or sulphur) within the ring which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonlamino and arylcarbonylamino.

Hypothermia: Lowering of the body temperature below normal level.

Ischemia: Restriction in blood supply with resultant dysfunction or damage of tissue.

Ischemic tissue damage: Tissue damage due to ischemia.

Moieties of a particular compound cover group(s) or part(s) of said particular compound.

Pharmaceutical composition: or drug, medicament or agent refers to any chemical or biological material, compound, or composition capable of inducing a desired therapeutic effect when properly administered to a patient. Some drugs are sold in an inactive form that is converted in vivo into a metabolite with pharmaceutical activity. For purposes of the present invention, the terms “pharmaceutical composition” and “medicament” encompass both the inactive drug and the active metabolite. Substituted lower alkyl means a lower alkyl having one to three substituents selected from the group consisting of hydroxyl, alkoxy, amino, amido, carboxyl, acyl, halogen, cyano, nitro and thiol.

The principle of the present invention is the use of cannabinoids and/or cannabimimetic compounds for induction of hypothermia for alleviating the effects of ischemia.

Ischemia

Ischemia is the reduction or abolition of blood supply to a tissue. The associated deficiency of oxygen and nutrients may lead to cell death (necrosis) in areas of the affected tissue. The damage induced by the lack of oxygenated blood in the brain occurs in two stages. First cellular metabolism is arrested due to lack of oxygen and some cells and tissue will die within minutes as a consequence hereof. Secondly a cascade of processes such as apoptosis are initiated and continue up to 12 hours after the event that initially induced the ischemic state has been abolished. The tissue damaged by the second cascade can be crucial and cause greater harm to the individual than the primary damage happening within the first minutes of ischemia.

The current invention is aimed at correcting ischemia of the brain thereby minimizing the damage to the central nervous system. The invention does so by administering a drug to induce hypothermia in patients. The hypothermic effect is presumed to counteract ischemic damage by several mechanisms in the brain: Prevention of the blood-brain-barrier disruption that happens soon after ischemic onset that allows oedema formation from extravasation; Diminishing of the oxygen-based free-radical production that results from activation of microglia and other cell types; Reduction of the excitotoxic-neurotransmitter release that overstimulates neighbouring neurons; Lowering of the metabolic rate and subsequent energy depletion; and anti-inflammatory action.

It is an object of the present invention to provide a compound capable of inducing hypothermia in an individual and further to provide the use of said compound for the production of a medicament for the treatment of ischemia in an individual.

Ischemia may occur under various circumstances; of special relevance to the present invention are the circumstances relating to cardiovascular diseases, asphyxia and traumatic brain injuries.

It is thus within the scope of the present invention to provide means for reducing the risk of ischemia as well as treating ischemia in an individual, under circumstances where ischemia is brought about by for example: cardiovascular diseases, asphyxia and traumatic brain injuries.

### Cardiovascular Diseases

Cardiovascular disease is the most common cause of death and of physical as well as mental impairment in the developed world. A similar development is seen in the rest of the world as it emulates the lifestyle of the Western hemisphere with its fatty diets, lack of exercise and increasing average lifespan.

The main causes of death and disability among cardiovascular diseases are myocardial infarction, acute coronary syndrome, cardiac arrest and stroke, but many less common cardiovascular diseases may be equally detrimental to the individual affected. These less common diseases include among others arterial aneurism, subarachnoid haemorrhage, arteriosclerosis, angina pectoris, hypertension, hypercholesterolemia, cardiac arrhythmia, cardiomegaly, cardiomyopathy, heart valve regurgitation and heart valve stenosis.

Each of the abovementioned diseases follow a course of events leading to ischemia, and are thus all of interest in relation to the present invention. Myocardial infarction (heart attack) is a result of an atherosclerotic. plaque slowly building up in the inner lining of a coronary artery which then suddenly ruptures, partially or totally occluding the artery and preventing blood flow. Cardiac arrest is the abrupt cessation of normal circulation of the blood due to failure of the heart to contract effectively. Brain damage is likely to occur after 3-4 minutes without medical intervention, except in cases of hypothermia. Stroke is an acute neurological injury, lasting more than 24 hours, in which the blood supply to a part of the brain is interrupted, either by a clot in the artery or if the artery bursts. Arterial aneurism is a localized ballooning of an artery by more than 50% of the diameter of the vessel. Aneurysms most commonly occur in the arteries at the base of the brain and in the aorta. This bulge in an artery carries the risk of bursting and leading to internal hemorrhage. The larger an aneurysm becomes, the more likely it is to burst. Subarachnoid haemorrhage (SAH) is bleeding into the subarachnoid space surrounding the brain, i.e., the area between the arachnoid and the pia mater. It may arise due to trauma or spontaneously, and is a medical emergency, which can lead to death or severe disability even if recognized and treated in an early stage. Arteriosclerosis is a disease in which arterial walls harden over years or decades as a result of the formation of collagen and calcium deposits. Hypertension or high blood pressure is a medical condition wherein the blood pressure is chronically elevated. Hypercholesterolemia is the presence of high levels of cholesterol in the blood. It is a derangement that can contribute to many forms of disease, most notably cardiovascular disease. Arrhythmia is a group of conditions in which the muscle contraction of the heart is irregular or is faster or slower than normal. Some arrhythmias are life threatening medical emergencies that can cause cardiac arrest and sudden death. Cardiomegaly is a medical condition wherein the heart is enlarged. It can often be associated with other serious medical conditions. Cardiomyopathy is the deterioration of the function of the myocardium (i.e., the actual heart muscle). People with cardiomyopathy are at risk of arrhythmia and/or sudden cardiac death. Heart valve regurgitation, also known as heart valve insufficiency, is the abnormal leaking of blood through heart valves. Heart valve stenosis is a heart condition caused by the incomplete opening of a heart valve, typically the aortic valve or the mitral valve, impairing blood flow through the heart.

Each of the cardiovascular diseases mentioned, as well as others not mentioned, may cause ischemia of organs. This ischemia, whether of the brain, heart or other organs, may lead to death or impairment if not treated rapidly.

It is an object of the present invention to provide a compound for the production of a medicament for the treatment or prophylaxis of an individual suffering from or at risk of suffering from of ischemia due cardiovascular diseases such as, but not limited to: myocardial infarction, acute coronary syndrome, cardiac arrest, stroke, arterial aneurism, subarachnoid haemorrhage, arteriosclerosis, angina pectoris, hypertension, hypercholesterolemia, cardiac arrhythmia, cardiomegaly, cardiomyopathy, heart valve regurgitation and heart valve stenosis.

Preferably, the medicament is for the treatment or prophylaxis of ischemia due to cardiac arrest, myocardial infarction, acute coronary syndrome, stroke, arterial aneurisms, sub-arachnoid haemorrhage or angina pectoris.

### Asphyxia

Asphyxia (suffocation) is a common cause of death and of physical as well as mental impairment in newborns, children and adults of all ages.

Asphyxia can be divided into perinatal asphyxia and non-perinatal asphyxia: Perinatal asphyxia is the medical condition resulting from deprivation of oxygen to a newborn infant long enough to cause apparent harm. It results most commonly from a drop in maternal blood pressure or interference during delivery with blood flow to the infants brain. This can occur due to inadequate circulation or perfusion, impaired respiratory effort, or inadequate ventilation. Extreme degrees of asphyxia can cause cardiac arrest and death. Hypoxic damage can occur to most of the infant's organs, but brain damage is of most concern and perhaps the least likely to quickly and completely heal. In severe cases, an infant may survive, but with damage to the brain manifested as developmental delay and spasticity; Non-perinatal asphyxia is a condition of severely deficient supply of oxygen to the body that arises from being unable to breathe normally. Common causes hereof include drowning, strangulation and exposure to toxic gasses. Asphyxia causes generalized hypoxia, which primarily affects the tissues and organs most sensitive to hypoxia first, such as the brain, hence resulting in cerebral hypoxia. The absence of effective remedial action will very rapidly lead to unconsciousness, brain damage and death.

Each kind of asphyxia mentioned, as well as others not mentioned, may cause ischemia of organs and is thus an object of the present invention.

It is an aspect of the present invention to provide a compound for the treatment of an individual suffering from ischemia due to asphyxia such as: perinatal asphyxia and/or non-perinatal asphyxia.

### Traumatic Brain Injury

Traumatic brain injury (TBI) is a common cause of death and of physical as well as mental impairment throughout the world. TBI may result from accidents, be due to violence or be self-inflicted.

Traumatic brain injury, also called intracranial injury, or simply head injury, occurs when a sudden trauma causes brain damage. TBI can result from a closed head injury or a penetrating head injury. Parts of the brain that can be damaged include the cerebral hemispheres, cerebellum, and brain stem. Symptoms of a TBI can be mild, moderate, or severe, depending on the extent of the damage to the brain. Outcome can be anything from complete recovery to permanent disability or death. lschemia is a significant factor contributing to the neurological damage frequently seen in patients suffering from TBI.

It is an aspect of the present invention to provide a compound for the treatment of an individual suffering from ischemia due to traumatic brain injury.

### Hypothermia

Hypothermia is the lowering of the core temperature of the body below normal level. Normal body temperature in an adult human measured rectally over 24 hours is 37 degree Celsius +/−0.6 degree Celsius and is thus variable between individuals, and over time within the individual. Hypothermia as a medical condition is usually defined as the effects seen on the body once the core temperature drops below 35 degree Celsius. It may become critical, if the body temperature falls below 32° C. In the present application hypothermia is defined as the lowering of the core body temperature below normal levels. This implies that any temperature below the normal core body temperature of the specific individual with its natural variations at the given point in time of the day, or period, herein is defined as being hypothermic. In particular, hypothermia is a temperature below 35.5° C., such as below 35° C., such as below 34.5° C., such as below 34.0° C.

Body temperature may be measured by a variety of means by mercury, electronic or plastic strip thermometers on different areas of the body such as the forehead, mouth, armpit, ear or rectum. It is presently understood, that the temperature referred to in the present application is the core body temperature, and that some of the above methods of measurement will indicate a different temperature than the core temperature.

It is of importance, that induction of hypothermia in an individual can follow a predictable course and be responsive to the dose in which the compound capable of inducing hypothermia is administered. The induction of the hypothermic condition may be rapid or slow depending on the situation of the individual in need of treatment. Also depending on the severity of the ischemic condition, it is of interest to provide a medicament for retaining the individual in the hypothermic state for variable durations of time. A single compound may be used depending on dosage within a range of temperatures or for the induction of hypothermia to a specific temperature. A combination of compounds may furthermore be used for an initial rapid decrease in core body temperature, and the subsequent maintenance of the reached temperature over a prolonged period. It is furthermore beneficial if the hypothermic state can be reversed in a rapid and controlled fashion either slowly or rapidly depending on the status of the individual.

It is thus an object of the present invention to provide a compound for the production of a medicament for the induction of hypothermia in an individual suffering from ischemia, wherein the compound is capable of inducing hypothermia to any range of temperatures between 37 and 31 degree Celsius, such as between 36.5 and 31.5 degree Celsius, such as between 36 and 32 degree Celsius, such as between 35.5 and 32.5 degree Celsius, such as between 35 and 33 degree Celsius, such as between 34.5 and 33.5 degree Celsius. The ranges may furthermore be between 37 and 34 degree Celsius, such as between 36.5 and 34.5 degrees, such as 36 and 35 degrees, alternatively between 34 and 31 degree, such as between 33.5 and 31.5 degree, or between 34 and 32 degree, such as 33 and 32 degree Celsius, alternatively between 36 and 33 degree or 35 and 32 degree Celsius. Preferably, the compound of the present is capable of inducing hypothermia in the range of between 36 to 32 degree Celsius, and more preferably between 35 and 33 degree Celsius.

It is also an object of the present invention to provide a compound capable of inducing hypothermia to a specific temperature such as 37 degree Celsius, 36.5 degree Celsius, 36 degree Celsius, 35.5 degree Celsius, 35 degree Celsius, 34.5 degree Celsius, 34 degree Celsius, 33.5 degree Celsius, 33 degree Celsius, 32.5 degree Celsius, 32 degree Celsius, 31.5 degree Celsius or 31 degree Celsius or most preferably, the compound of the present invention is capable of inducing hypothermia to any of the above specific temperatures within a range of +/−0.5 degree Celsius, the range thus being between +/−0.4 degree Celsius, such as between +/−0.3 degree Celsius, such as between +/−0.2 degree Celsius, or such as between +/−0.1 degree Celsius. The temperature range or specific temperature a given compound is capable of inducing is herein also referred to as the target temperature of the compound and/or the medicament comprising the compound.

### Cannabinoids

Cannabinoids and cannabimimetic compounds are a group of chemicals which activate the body's cannabinoid receptors, CB1 and CB2. Before other types were discovered, the term referred to a unique group of secondary metabolites found in the cannabis plant, which are responsible for the plant's peculiar pharmacological effects. Cannabinoids are generally grouped into five classes based mainly on chemical composition and in part on origin:
1. The eicosanoids, also referred to as endocannabinoids are produced in the bodies of humans and other animals
2. Classical cannabinoids, a group which includes natural cannabinoids found in larger or smaller amounts in the hemp plant Cannabis sativa.
3. Non-classical cannabinoids
4. Aminoalkylindoles
5. Other compounds that are capable of binding cannabinoid receptors, but fall out of the four previous categories.

The current understanding recognizes the role that endocannabinoids play in almost every major life function in the human body. Cannabinoids act as a bioregulatory mechanism for most life processes, which reveals why medical cannabis has been cited as treatments for many diseases and ailments in anecdotal reports and scientific literature. Some of these ailments include: pain, arthritic conditions, migraine headaches, anxiety, epileptic seizures, insomnia, loss of appetite, GERD (chronic heartburn), nausea, glaucoma, AIDS wasting syndrome, depression, bipolar disorder (particularly depression-manic-normal), multiple sclerosis, menstrual cramps, Parkinson's, trigeminal neuralgia (tic douloureux), high blood pressure, irritable bowel syndrome, and bladder incontinence. Cannabinoids and cannabimimetic compounds (CB1/CB2 agonists) have furthermore received interest as putatively neuro-protective substances.

Several mechanisms have been proposed to account for the neuroprotective effects of various cannabinoids and cannabimimetic substances such as prevention of excitotoxicity by cannabinoid CB1-mediated inhibition of glutamatergic transmission, reduction of calcium influx, anti-oxidant activity, activation of the phosphatidylinositol 3-kinase/protein kinase B pathway, induction of phosphorylation of extracellular regulated kinases and the expression of transcription factors and neutrophins, lowering of the cerbrovasoconstriction and induction of hypothermia.

Any compound which can be defined as a cannabinoid or cannabimimetic compound falls within the scope of the present invention. The two terms cannabinoids and cannabimimetic are used interchangeably herein. Cannabinoids are generally termed such due to their ability to bind one or more of the cannabinoid receptors CB1 and CB2.

### Receptors

There are currently two known types of cannabinoid receptors, CB1 and CB2, which are common in animals, and have been found in mammals, birds, fish, and reptiles.

CB1 receptors are found primarily in the brain, specifically in the basal ganglia and in the limbic system, including the hippocampus. They are also found in the cerebellum and in both male and female reproductive systems. CB1 receptors are essentially absent in the medulla oblongata, the part of the brain that is responsible for respiratory and cardiovascular functions. Thus, there is not a risk of respiratory or cardiovascular failure as there is with many other drugs. CB1 receptors appear to be responsible for the euphoric and anticonvulsive effects of cannabis.

CB2 receptors are almost exclusively found in the immune system, with the greatest density in the spleen. CB2 receptors appear to be responsible for the anti-inflammatory and possible other therapeutic effects of cannabis.

Researchers have noted that the behavioural effects, including hypothermia, seen when introducing animals to cannabinoids seems to be due to other factors besides CBI receptor stimulation [37;40]. Inducing hypothermia by cannabinoids is therefore not solely equivalent to stimulating the CBI receptor. Furthermore, there is evidence in the literature for other receptors than CB1 and CB2 as recipients of the cannabinoid ligands.

The receptors to which the cannabinoids and cannabimimetic compounds of the present invention may bind includes, apart from CB1 and CB2: a third CB receptor, herein termed CB3, GABA (gamma-aminobutyric acid) receptors, the NMDA (N-methyl-D-aspartate) receptor, the 5-HT(1A) receptor, also known as the serotonin receptor, the Delta opioid receptor (DOR) and TRPV1 (transient receptor potential vanilloid 1). It is furthermore within the scope of the invention that the compounds of the invention may bind CB1, CB2 or CB3 co-receptors. Compounds capable of binding any of the above-mentioned receptors thus fall within the scope of the present invention.

### Structure

The cannabinoids of this application are, based on their structure, categorized as follows: classic cannabinoids, non-classic cannabinoids, aminoalkylindoles, eicosanoids (endogenous cannabinoids) and other compounds that fall out of the classification. Compounds belonging to any of these categories fall within the scope of the present invention.

It is within the scope of the invention that the compounds of the invention are capable of inducing hypothermia in an individual.

It is furthermore within the scope of the invention that the compounds of the invention are capable of binding a cannabinoid receptor.

Accordingly, in the broadest aspect the present invention concerns the use of a compound comprising a structure of one of the general formulas illustrated in the below. In these illustrations R is a chemical bond or a chemical moiety as defined in the above. R may be any moiety substituted any amount of times according to the following non-limiting list, whereby R is: C, H, S, N, O, optionally substituted one or more times with C, H, S, N, O, P, OH, hydrogen, alkyl, alkenyl, alkynyl, phenyl, diphenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl or phosphate, optionally further substituted one or more times with C, S, N, O, P, OH, H, phenyl, amine (NH), halogen, substituted lower alkyl or alkyl such as (C₁-Cₓ) any of which may be further substituted one or more times with methyl, dimethyl, alkyl such as (C₁-Cₓ), phenyl, sulphate, phosphate, halogen or further substituted by fluoride, sulphate, phosphate, methyl, dimethyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, dicycloalkyl, tricycloalkyl, cycloalkenyl, alkoxy, carboxy, halogen, trifluoromethyl, cyano, amino, nitro, halogen or alcohol, and wherein x is an integer of from 1 to 30, and of which any of the mentioned substituents capable thereof may form a ring with another R, R may furthermore be a chemical bond, or a pharmaceutically acceptable addition salt or hydrate thereof.

Phosphate resides have been implicated in the reduction of toxicity of certain cannabinoids without altering their hypothermic effect. It is therefore an object of the present invention that any of the compounds may carry one or more phosphate groups bound as phosphate esters.

For each general formula, a more specific choice of substituent for a given R is listed along with along with a preferred and a more preferred list of substituent groups.

The present invention concerns the use of a compound such as a classic or non-classic cannabinoid comprising the general formula (I):

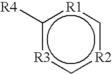

wherein R1 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl or phosphate, optionally further substituted one or more times with C, S, N, O, OH, phenyl, amine (NH), halogen, methyl, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, and preferably is C, O, N optionally substituted with O, OH, alkyl, alkenyl, alkynyl, or phosphate, optionally further substituted with methyl, alkyl or phosphate and more preferably is C, optionally substituted with H, OH, OCH₃ or phosphate and
wherein R2 is selected from the group of: C, S, N, O, optionally substituted with C, S, N, O, P, OH, hydrogen, alkyl, alkenyl, alkynyl, any of which may or may not be branched or comprise substituents such as phosphate, cycloalkyl, heterocycloalkyl, cycloalkenyl, methyl, dimethyl, or may be further substituted one or more times with C, S, N, O, P, OH, hydrogen, alkyl, alkenyl, alkynyl, sulfonyl, any of which may or may not be branched or comprise substituents such as hydrogen, alkyl, alkenyl, alkynyl, fluoride, phosphate, cycloalkyl, heterocycloalkyl, cycloalkenyl, dimethyl, phenyl and preferably is C substituted with C, O, P, H, OH, OSO₂, phosphate, alkyl, alkenyl, alkynyl such as (C₁-Cₓ), phenyl any of which may be substituted with methyl, dimethyl, sulfonyl, cycloalkyl, heterocycloalkyl, cycloalkenyl, fluoride, phenyl, phosphate, and more preferably is C substituted with C, O, OSO₂, alkyl such as (C₃-C₁) any of which may be further substituted with methyl, dimethyl, alkyl such as (C₁-Cₓ), phenyl, phosphate or further substituted by fluoride, phosphate, methyl, dimethyl and wherein x is an integer of from 1 to 20 and
wherein R3 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkenyl, phosphate, optionally further substituted one or more times with C, S, N, O, OH, methyl, phenyl, diheterocycle, amine (NH), halogen, substituted lower alkyl, aryl, lower alcohol, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, preferably is C, O, N, S, optionally substituted with O, OH, alkyl, alkenyl, alkynyl, or phosphate, optionally further substituted one or more times with methyl, diheterocycle, lower alcohol, alkyl or phosphate more preferably is C which may be substituted with C, O, N, OH, phosphate, any of which may be substituted one or more times with C, ethyl, methyl, phosphate, diheterocycle, lower alcohol, alkyl such as (C₁-C₂) wherein C₂ binds to R4 when R4 is C, optionally further substituted by methyl, dimethyl or phosphate and
wherein R4 is selected from the group of: C, H, S, N, O, optionally substituted with C, H, S, N, O, P, OH, hydrogen, alkyl, alkenyl, alkynyl, phenyl, diphenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally further substituted one or more times with C, S, N, O, OH, phenyl, amine (NH), halogen, substituted lower alkyl, alkyl such as (C₁-Cₓ) any of which may be further substituted with methyl, dimethyl, alkyl such as (C₁-Cₓ), phenyl, phosphate or further substituted by fluoride, phosphate, methyl, dimethyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, dicycloalkyl, tricycloalkyl, cycloalkenyl, alkoxy, carboxy, halogen, trifluoromethyl, cyano, amino, or nitro, (alcohol) and preferably is C, H, N, O optionally substituted with alkyl, alkenyl, alkynyl, alcohol, phenyl, diphenyl, dicycloalkyl, tricycloalkyl, cycloalkenyl any of which may bond with R1 or R3 forming a ring, optionally further substituted with one or more alkyl, alkenyl, alkynyl, OH, and more preferably is C, H, (C1-C_{y}), dicycloalkyl, or tricycloalkyl, cycloalkenyl any C of which may bond with R1 or R3 forming a ring, and optionally is substituted with methyl, dimethyl, phenyl, diphenyl, optionally further substituted with alkyl and/or OH and wherein x is an integer of from 1 to 15 and y is an integer of from 1 to 8.

Preferably, the present invention concerns the use of a compound comprising the general formula (I) wherein R1 is C, O, N optionally substituted with O, OH, alkyl, alkenyl, alkynyl, or phosphate, optionally further substituted with methyl, alkyl or phosphate, when R2 is C substituted with C, O, P, H, OH, OSO2, phosphate, alkyl, alkenyl, alkynyl such as (C1-CX), phenyl any of which may be substituted with methyl, dimethyl, sulfonyl, heterocycloalkyl, fluoride, phenyl or phosphate, when R3 is C, O, N, S, optionally substituted with O, OH, alkyl, alkenyl, alkynyl, or phosphate, optionally further substituted one or more times with methyl, diheterocycle, lower alcohol, alkyl or phosphate, when R4 is C, H, N, O optionally substituted with alkyl, alkenyl, alkynyl, alcohol, phenyl, diphenyl, dicycloalkyl, tricycloalkyl, cycloalkenyl any of which may bond with R1 or R3 forming a ring, optionally further substituted with one-or more alkyl, alkenyl, alkynyl or OH.

Most preferably, the present invention concerns the use of a compound comprising the general formula (I) wherein R1 is C, optionally substituted with H, OH, OCH₃ or phosphate, when R2 is C substituted with C, O, OSO₂, alkyl such as (C₃-C₁₁) any of which may be further substituted with methyl, dimethyl, alkyl such as (C₁-Cₓ), phenyl, phosphate or further substituted by fluoride, phosphate, methyl, dimethyl when R3 is C which may be substituted with C, O, N, OH, phosphate, any of which may be substituted with C, ethyl, phosphate, alkyl such as (C₁-C₂) wherein C₂ binds to R4 when R4 is C, optionally further substituted by methyl, dimethyl or phosphate when R4 as defined in claim 8 is C, (C1-C8) any C of which may bond with R3 and optionally is substituted with methyl, dimethyl, phenyl, diphenyl optionally further substituted with an alcohol and wherein x is an integer of from 1 to 15.

The present invention also concerns the use of a compound such as a classic or non-classic cannabinoid comprising the general formula (II):

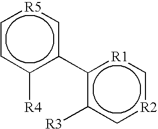

wherein R1 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally bonding with the C in the ring next to R5, optionally further substituted one or more times with C, S, N, O, OH, phenyl, phosphate, amine (NH), halogen, methyl, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, and preferably is C, O, N optionally substituted with O, OH, alkyl, alkenyl, alkynyl, or phosphate, optionally further substituted with alkyl or phosphate and more preferably is C, optionally substituted one or more times with H, O, OH, OCH₃ or phosphate and
wherein R2 is selected from the group of: C, S, N, O, optionally substituted with C, S, N, O, P, OH, hydrogen, alkyl, alkenyl, alkynyl, any of which may or may not be branched or comprise substituents such as phosphate, heterocycloalkyl, cycloalkyl, cycloalkenyl, methyl, dimethyl, or may be further substituted with C, S, N, O, P, OH, hydrogen, alkyl, alkenyl, alkynyl, sulfonyl, any of which may or may not be branched or comprise substituents such as hydrogen, alkyl, alkenyl, alkynyl, fluoride, phosphate, heterocycloalkyl, cycloalkyl, cycloalkenyl, dimethyl, phenyl and preferably is C substituted with C, O, P, H, OH, OSO₂, phosphate, alkyl, alkenyl, alkynyl such as (C₁-Cₓ), phenyl any of which may be substituted with methyl, dimethyl, sulfonyl, heterocycloalkyl, fluoride, phenyl, phosphate, and more preferably is C substituted with C, O, OSO2, alkyl such as (C₃-C₁₁) any of which may be further substituted with methyl, dimethyl, alkyl such as (C₁-Cₓ), phenyl, phosphate or further substituted by fluoride, phosphate, methyl, dimethyl and wherein x is an integer of from 1 to 15, and
wherein R3 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, any of which may connect with R4 and preferably is C, O, N, OH, phosphate optionally substituted one or more times with alkyl, OH, phosphate any of which may connect with R4 and more preferably is O, OH, NH, optionally connecting with R4 thus forming a ring and
wherein R4 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, any of which may connect with R3 and preferably is C, N, O, P, OH, lower substituted alkyl, alkenyl, alkynyl, phenyl, optionally substituted with OH, methyl, dimethyl any of which may connect with R3 and more preferably is C, optionally connecting with R3 and optionally substituted with methyl, dimethyl or methyn and
wherein R5 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally bonding with R1, optionally further substituted one or more times with C, S, N, O, OH, phenyl, amine (NH), halogen, methyl, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, phosphate or nitro, and preferably is C, N, O, optionally substituted with C, O, CH2OH, methyl, dimethyl, alkyl, alkenyl, alkynyl, phenyl, phosphate and more preferably is C, CO, optionally substituted with C, methyl, methyn (CH2), optionally substituted with CH2OH.

Preferably, the present invention concerns the use of a compound comprising the general formula (II) wherein R1 is C, O, N optionally substituted with O, OH, alkyl, alkenyl, alkynyl, or phosphate, optionally further substituted with alkyl or phosphate, when R2 is C substituted with C, O, P, H, OH, OSO2, phosphate, alkyl, alkenyl, alkynyl such as (C1-CX), phenyl any of which may be substituted with methyl, dimethyl, sulfonyl, heterocycloalkyl, fluoride, phenyl or phosphate, when R3 is C, O, N, OH, phosphate optionally substituted one or more times with alkyl, OH, phosphate any of which may connect with R4 thus forming a ring, when R4 is C, N, O, P, OH, lower substituted alkyl, alkenyl, alkynyl, phenyl, optionally substituted one or more times with OH, methyl and/or dimethyl any of which may connect with R3, when R5 is C, N, O, optionally substituted with C, O, CH2OH, methyl, dimethyl, alkyl, alkenyl, alkynyl, phenyl or phosphate.

Most preferably, the present invention concerns the use of a compound comprising the general formula (II) wherein RI is C, optionally substituted with H, OH, OCH₃ or phosphate when R2 is C substituted with C, O, OSO2, alkyl such as (C₃-C₈) any of which may be further substituted with methyl, dimethyl, alkyl such as (C₁-Cₓ), phenyl, phosphate or further substituted by fluoride, phosphate, methyl, dimethyl when R3 is O, OH, NH, optionally connecting with R4, when R4 is C, optionally connecting with R3 and optionally substituted with methyl, dimethyl or methyn, when R5 is C, CO, optionally substituted with C, methyl, methyn (CH2), optionally substituted with CH2OH and wherein x is an integer of from 1 to 15.

In relation to the classic and non-classic cannabinoids and cannabimimetic compounds illustrated here by the general formulas (I) and (II), the presence of a phenolic hydroxyl group seems to play an essential role for ensuring high affinity binding of the compounds to the cannabinoid receptors.

An additional element of importance for especially CB1 receptor recognition is the side chain of R2. It is preferably a lipophilic carbon chain comprising from 1 to 15 carbon atoms, preferably from 3 to 11 carbon atoms. It may have any number and type of substituents, especially methyl and/or dimethyl groups. The methyl groups are preferably close to the phenol group, as this appears to induce the greatest effect of the drug. Interestingly, it appears that shorter side chains increase the intensity and decrease the duration of the activity of the compounds.

The present invention concerns the use of a compound such as an eicosanoids or other cannabinoid compound comprising the general formula (III):

R1—R2
wherein R1 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, and preferably is (C1-Cx) is saturated or unsaturated and optionally is substituted one or more times with lower alkyl, alkenyl, alkynyl, O, OH, N and wherein x is an integer of from 1 to 30, more preferably is (C1-CY), is saturated or unsaturated and optionally substituted with methyl, dimethyl, O, or N and wherein Y is an integer of from 15 to 21 and
wherein R2 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally further substituted one or more times with C, S, N, O, OH, phenyl, amine (NH), halogen, methyl, OCH3, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, and preferably is C, N, O, NH2 optionally substituted one ore more times with lower alkyl, alkenyl, alkynyl, phenyl, OH, NH2 cycloalkane, methyl, OCH3, and more preferably is N, O, NH2 optionally substituted with C, CH2OH, CH(CH2)2, C2H4, C3H6, optionally further substituted one or more times with NH2, OH, CH2OH, CH2Cl, phenyl, CH3 and/or OCH3.

Preferably, the present invention concerns the use of a compound comprising the general formula (III) wherein R1 is (C1-Cₓ) saturated or unsaturated, and optionally is substituted one or more times with lower alkyl, alkenyl, alkynyl, O, OH, N, when R2 is C, N, O, NH2 optionally substituted one ore more times with lower alkyl, alkenyl, alkynyl, phenyl, OH, NH2 cycloalkane, methyl or OCH3 and wherein ₓ is an integer of from 1 to 30.

Most preferably, the present invention concerns the use of a compound comprising the general formula (III) wherein R1 is (C₁-Cₓ), is saturated or unsaturated and optionally substituted with methyl, dimethyl, O, or N when R2 is N, O, NH2 optionally substituted with C, CH2OH, CH(CH2)2 (cyclopropane), optionally further substituted one or more times with CH2OH, CH2Cl and wherein x is an integer of from 1 to 21.

The present invention concerns the use of a compound such as an aminoalkylindole or other cannabinoid compound comprising the general formula (IV):

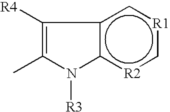

wherein R1 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, and preferably is C, O, N optionally substituted with O, phosphate, N, C, lower alkyl, OH, optionally further substituted with lower alkyl, OH, phosphate and more preferably is C, substituted with O, further substituted with methyl and
wherein R2 is selected from the group of; C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, any of which may bond with R3, and preferably is C, N, O, optionally substituted with C, O, N, phosphate, lower alkyl optionally further substituted with lower alkyl, OH, phosphate, any of which may bond with R3 and more preferably is C, substituted with O, further substituted with C optionally bond forming with R3 and
wherein R3 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, any of which may bond R2 and preferably is C, N, O, alkyl, alkenyl, alkynyl, optionally substituted with C, N, O, OH, phosphate, halogen any of which may bond R2 and more preferably is (C1-Cx) and wherein x is an integer of from 1 to 3, optionally substituted one or more times with O, dichloro-phenyl or morpholine and any of which may bond R2 and
wherein R4 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C₁₋₄alkyl, heteroaryl-(C₁₋₄)-alkyl, heterocyclyl-(C₁₋₄)-alkyl, cycloalkylalkyl, cycloalkyl, cycloalkenyl, alkoxy, carboxy, halogen, trifluoromethyl, cyano, amino, or nitro, and preferably is C, N, O optionally substituted with C, N, O, OH, lower alkyl, alkenyl, alkynyl, phosphate, optionally further substituted one or more times with O, OH, phenyl, diphenyl, morpholino, and halogen, and more preferably is C, optionally substituted with C, O and/or diphenyl, optionally further substituted with morpholine.

Preferably, the present invention concerns the use of a compound comprising the general formula (IV) wherein R1 is C, O, N optionally substituted with O, phosphate, N, C, lower alkyl, OH, optionally further substituted with lower alkyl, OH or phosphate, when R2 is C, N, O, optionally substituted with C, O, N, phosphate, lower alkyl optionally further substituted with lower alkyl, OH, phosphate, any of which may bond with R3, when R3 is C, N, O, alkyl, alkenyl, alkynyl, optionally substituted with C, N, O, OH, phosphate, halogen any of which may bond R2, when R4 is C, N, O optionally substituted with C, N, O, OH, lower alkyl, alkenyl, alkynyl, phosphate, optionally further substituted one or more times with O, OH, phenyl, diphenyl, morpholino, and/or halogen.

Most preferably, the present invention concerns the use of a compound comprising the general formula (IV) wherein, wherein R1 is C, substituted with O, further substituted with methyl when R2 is C, substituted with O, further substituted with C optionally bond forming with R3 when R3 is (C1-Cx) and wherein x is an integer of from 1 to 3, optionally substituted one or more times with O, dichloro-phenyl or morpholine when R4 is C, optionally substituted with C, O and/or diphenyl, optionally further substituted with morpholine.

### Examples of Compounds

Examples of compounds specially relevant for the present invention include, but is not limited to classic cannabinoids such as (names in parenthesis are alternative names): delta-9-THC (Tetrahydrocanna-binol), delta-8-THC, delta-8-THC phosphate, Cannabinol (CBN), Cannabidiol (CBD), Cannabidiol-type CBD, Cannabidivarin (CBDV), Cannabichromene-type CBG, Cannabigerol-type CBG, Tetrahydrocanna-bivarin (THCV, THV), Tetrahydrocanna-binol- and cannabinol-type THC or CBN, Iso-Tetrahydro-cannabinol-type iso-THC, Cannabielsion-type CBE, Cannabicyclo I-type CBL, Cannabicitran-type CBT, HU-308, JWH-133, JWH-139, JWH-051, L-759633, L-759656, HU-210 ((−)-11-OH-delta-8-tetrahydrocannabinoldimethylheptyl), HU-211 (Dexanabinol, 7-hydroxy-□6-tetrahydrocannabinol 1,1-dimethylheptyl), Desacetyl-L-nandrolol, Nabilone and Levonantradol, non-classic cannabinoids such as: CP-55940, CP55244 and CP47497, aminoalkylindoles such as: R(+)WIN55212, S(-)WIN-55213, JWH-015 and L-768242, eicosanoids/endogenous cannabinoids such as: Anandamide (arachidonyl ethanolamine), 2-Arachidonyl-glycerol (2-AG, Noladin ether), Palmitoylethanol-amine, Virodhamine (O-arachidonoyl-ethanolamine), Palmitoyl ethanolamide, Oleamide, other cannabinoid compounds such as: Arvanil, Metanandamide, ACEA, ACPA, BAY 38-7271 and O-1812. Phosphate derivatives of these compounds are especially relevant for the present invention.

Examples of especially relevant compounds are anandamide, delta-9-THC, delta-8-THC, cannabidiol, HU-210, BAY 38-7271, WIN 55,212 and CP55940 and the phosphate derivatives of these.

### Preferred Compound

The compounds of the present invention may apart from inducing hypothermia, induce secondary effects or have other characteristics. These may be related to the cannabinoid nature of the compounds and may thus be more or less desirable. It is preferable that the compounds of the invention do not induce any adverse psychotropic effects. The compound may furthermore have analgesic, anti-convulsive, anti-inflammatory, anti-anxiety, anti-nausea, pulse-lowering and blood-pressure modifying effects. Of these, it is preferable that the compound has analgesic effects. Furthermore, a compound of the present invention may be hydrophilic or hydrophobic. To facilitate the administration of a compound according to the present invention it is preferable for a compound to be hydrophilic. A preferred compound is moreover metabolically stable.

A preferred compound of the present invention is a compound capable of binding a cannabinoid receptor, such as CB1, thereby inducing hypothermia in an individual to a temperature in the range of 36 to 32 degree Celsius, and where said compound is hydrophilic.

### Antagonists

It is an object of the present invention to provide compounds that are capable of obviating the effect of the compounds that induce hypothermia. These compounds are herein termed antagonists and exert their antagonistic effect by blocking the ability of any of the cannabinoids or cannabimimetic compounds herein described in binding to their receptors. The purpose of such an antagonist is to provide an additional safety mechanism whereby it is possible to stop the decline in core body temperature, stabilize the core body temperature and/or raise the core body temperature of an individual.

An embodiment of the present invention thus comprises the use of a compound according to any of the above for the preparation of a medicament for antagonizing the induction of hypothermia in an individual.

Examples of antagonists includes but is not limited to: Rimonabant (SR141716, Acomplia, SR147778, SR141716A, SR144528, CP-272,871, NIDA-41020, LY320135, AM251, AM281, AM630, WIN56098 and WIN54461.

### Novel Use of Compounds

Cannabinoids and cannabimimetic compounds have been used for a variety of purposes over time. It is an object of the present invention to provide a novel use of these compounds for the induction of hypothermia, especially for the induction of hypothermia in an individual suffering from ischemia or at risk of suffering from ischemia.

### Medicament

The induction of hypothermia by any of the herein described compounds is performed by preparing, producing and thus providing a medicament or pharmaceutical composition comprising at least one of said compounds. The medicament of the present invention is thus for the induction of hypothermia in an individual for the treatment and/or prophylaxis of ischemia in said individual.

### Pharmaceutical Composition

Whilst it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier there for.

The compounds of the present invention may be formulated in a wide variety of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, wetting agents, tablet disintegrating agents, or an encapsulating material.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulphates, and sulfosuccinates, (c) non-ionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimize or eliminate irritation at the site of injection, such compositions may contain one or more non-ionic surfactants having a hydrophile-lipophile balance (HLB) of from about 12 to about 17. The quantity of surfactant in such formulations will typically range from about 5to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Pharmaceutically Acceptable Salts

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

A pharmaceutically acceptable salt means any salt of the compounds mentioned. In particular, it means a pharmaceutically acceptable acid addition salt. Pharmaceutically acceptable acid addition salts of the compounds include salts derived from non-toxic inorganic acids such as hydrochloric, nitric, phosphoric, sulphuric, hydrobromic, hydriodic, hydrofluoric, phosphorous and the like, as well as the salts derived from non-toxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulphate, pyrosulphate, bisulphate, sulphite, bisulphite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like.

pH

An aspect of the present invention regards the pH of the medicament. The pH of the medicament depends on the administration form, as the pH of the medicament preferably is suitable for the route of administration chosen. An embodiment of the present invention comprises a medicament wherein the pH of the composition is between pH 5 and pH 9, such as between pH 5.5 and 8.5 such as between pH 6 and 8, such as between pH 6.5 and 7.5. Most preferably the pH of the medicament is in accordance with the route of administration and the tissue to which the medicament is administered.

### Indications

The invention provides compounds for the production of a medicament for the treatment of ischemia in an individual. Ischemia may arise due to various circumstances and thus it is an object of the present invention to treat ischemia arising from a plurality of medical indications.

These indications include, but are not limited to, cardiovascular diseases such as myocardial infarction, acute coronary syndrome, cardiac arrest, stroke, arterial aneurism, subarachnoid haemorrhage, arteriosclerosis, angina pectoris, hypertension, hypercholesterolemia, cardiac arrhythmia, cardiomegaly, cardiomyopathy, heart valve regurgitation and heart valve stenosis, perinatal asphyxia and non-perinatal asphyxia as well as traumatic brain injury.

### Target Temperature

The target temperature of the medicament is the core body temperature that can be - reached upon administering the medicament according to the present invention as prescribed according to potency, dosage and so on. Various ranges and specific hypothermic core body temperatures that fall within the scope present invention are equal to the temperatures that the compound itself may induce as listed in the section on hypothermia.

An embodiment of the present invention is thus a medicament capable of inducing hypothermia below 36° C., such as below 35.5° C., such as below 35° C., such as below 34.5° C., such as below 34.0° C. in the range of between 36 to 32 degree Celsius, and more preferably between 35 and 33 degree Celsius.

### Administration

The main routes of drug delivery, in the treatment method are intravenous, oral, and topical, as will be described below. Other drug-administration methods, such as subcutaneous injection or via inhalation, which are effective to deliver the drug to a target site or to introduce the drug into the bloodstream, are also contemplated.

The mucosal membrane to which the pharmaceutical preparation of the invention is administered may be any mucosal membrane of the individual to which the biologically active substance is to be given, e.g. in the nose, vagina, eye, mouth, genital tract, lungs, gastrointestinal tract, or rectum, preferably the mucosa of the nose, mouth or rectum.

Compounds of the invention may be administered parenterally, that is by intravenous, intramuscular, intraspinal, subcutaneous, intranasal, intrarectal, intravaginal or intraperitoneal administration. The subcutaneous and intramuscular forms of parenteral administration are generally preferred. Appropriate dosage forms for such administration may be prepared by conventional techniques. The compounds may also be administered by inhalation, that is by intranasal and oral inhalation administration. Appropriate dosage forms for such administration, such as an aerosol formulation or a metered dose inhaler, may be prepared by conventional techniques.

The compounds according to the invention may be administered with at least one other compound. The compounds may be administered simultaneously, either as separate formulations or combined in a unit dosage form, or administered sequentially.

A preferred embodiment of the present invention is a medicament for administration by injection, suppository, oral administration, sublingual tablet or spray, cutaneous administration, or inhalation. More preferably the administration form is by injection, wherein the injection is intravenous, intramuscular, intraspinal, intraperitoneal, subcutaneous, a bolus or a continuous administration.

It has previously been demonstrated that administration of cannabinoids by the intravenous route produced a greater hypothermic response than that produced by the intraperitoneal route, [41]. Therefore the most preferable mode of administration of a medicament comprising a compound according to the present invention for the induction of hypothermia in an individual suffering from ischemia is by intravenous injection.

### Individual

The individual that may benefit from the administration of a medicament as described herein may be an individual suffering from ischemia or at risk of suffering from ischemia. If the individual is at risk of suffering from ischemia the preferred administration form of the medicament may be suppository, oral administration or inhalation. Preferably, the individual is an individual suffering from ischemia. The preferred administration form for an individual suffering from ischemia is an injection, such as an intravenous, intramuscular, intraspinal, intraperitoneal or subcutaneous injection.

The individual may be any human being, male or female, infant or old. The ischemic condition to be treated or prevented in the individual may relate to the age of the individual, the general health of the individual and whether or not the individual has a prior history of suffering from diseases or disorders that may have or have induced ischemic conditions in the individual.

### Dosage

The dosage of the compound according to the invention depends on the compound in question; however, the amount of the compound is also closely related to the pharmaceutical composition of the medicament, any second compound of the medicament or any second active ingredient of the medicament.

For all methods of use disclosed herein for the compounds, the daily oral dosage regimen will preferably be from about 0.01 to about 80 mg/kg of total body weight.

The daily parenteral dosage regimen will be from about 0.001 to about 80 mg/kg of total body weight.

For all methods of use disclosed herein for the compounds, the daily oral dosage regimen will preferably be from about 0.01 to about 80 mg/kg of total body weight. The daily parenteral dosage regimen will be from about 0.01 to about 2,400 mg/kg of total body weight, preferably, the dosage of the medicament according to the present invention is between 10 μg to 10 mg pr kg total body weight, such as between 100 μg to 1 mg pr kg total body weight, depending on the cannabinoid of choice. It has been found that cannabinoids varies with respect to potency and affinity for the cannabinoid receptor as well as with respect to molecular weight.

For one of the compounds in this invention, HU-210, the dosage regime will be between 2 and 1000 microgram/kg of total body weight, such as between 4 and 900 microgram/kg of total body weight, such as between 6 and 800 microgram/kg of total body weight, such as between 8 and 700 microgram/kg of total body weight, such as between 10 and 600 microgram/kg of total body weight. Preferably, the dosage regime will be between 15 and 500 microgram/kg of total body weight, more preferably between 20 and 400 microgram/kg of total body weight. More preferably at least 40 microgram/kg of total body weight, such as at least 50 microgram/kg of total body weight, such as at least 60 microgram/kg of total body weight, such as at least 70 microgram/kg of total body weight, such as at least 80 microgram/kg of total body weight, such as at least 100 microgram/kg of total body weight. The dosages mentioned are the dosages for inducing hypothermia as defined herein. The dosage may be administered as one single bolus or divided into two or more dosages given over a period of time. Additionally the hypothermic effect may be maintained by administering one or more dosages some hours after the first dosage, such as at least 6 hours later, or such as at least 12 hours later. Such additionally dosages may be of the same amount as the first dosage or an amount being at the most one-half or one-quarter of the first dosage.

For another compound in this invention, delta-8-THC phosphate, the dosage regime will be between 0.25 and 600 mg/kg of total body weight, such as between 0.5 and 500 mg/kg of total body weight, 1 and 400 mg/kg of total body weight, 2 and 300 mg/kg of total body weight, 3 and 200 mg/kg of total body weight. Preferably, the dosage regime will be between 4 and 150 mg/kg of total body weight, more preferably between 5 and 100 mg/kg of total body weight. More preferably at least 20 mg/kg of total body weight, such as at least 30 mg/kg of total body weight, such as at least 40 mg/kg of total body weight, such as at least 50 mg/kg of total body weight, such as at least 60 mg/kg of total body weight, such as at least 100 mg/kg of total body weight. The dosages mentioned are the dosages for inducing hypothermia as defined herein. The dosage may be administered as one single bolus or divided into two or more dosages given over a period of time. Additionally the hypothermic effect may be maintained by administering one or more dosages some hours after the first dosage, such as at least 6 hours later, or such as at least 12 hours later. Such additionally dosages may be of the same amount as the first dosage or an amount being at the most one-half or one-quarter of the first dosage.

For any other cannabinoid receptor agonist compound according. to the invention the exact dosage may be calculated based on the porcine study model described in Example 5.

The term “unit dosage form” as used herein refers to physically discrete units suitable as unitary dosages for human and animal individuals, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the individual that corresponds to a concentration of one or more compounds according to the invention. Also, the effective level depends on the severity of the ischemic condition, such as total amount of tissue experiencing hyp- or anoxia, the duration of the ischemic condition, whether it is the first or a subsequent ischemic attack of the individual and so forth.

### Dosage Regime and Duration of Treatment

The medicament may be administered in any suitable dosage regime, suitable as to the potency of the compound/drug, the target temperature to be reached, the speed of action of the compound, the metabolic stability of the compound, the duration of the treatment and how often the medicament optimally is to be administered.

It is within the scope of the invention to provide a medicament to be administered at intervals of 30 minutes to 24 hours, such as intervals of 1 to 23 hours, 2 to 22 hours, 3 to 20 hours, 4 to 18 hours, 5 to 16 hours, 6 to 14 hours, 7 to 12 hours or 8 to 10 hours. Preferably, the administration occurs at intervals of I to 6 hours, such as 2 to 5 hours, 3 to 4 hours.

The optimal administration interval depends on the duration of the hypothermic treatment. The duration of the treatment depends among other things on the severity of the ischemic condition. It is within the scope of the present invention to provide medicaments for the induction of hypothermia wherein the duration of the treatment is from 6 to 72 hours, such as from 7 to 69 hours, such as from 8 to 66 hours, 9 to 63 hours, 10 to 60 hours, 11 to 57 hours, 12 to 54 hours, 13 to 51 hours, 14 to 48 hours, 15 to 45 hours, 16 to 42 hours, 17 to 39 hours, 18 to 36 hours, 1 to 35 hours, 20 to 32 hours, 21 to 29 hours, 22 to 26 hours 23 to 25 hours. Preferably, the duration of the treatment is between 6 and 48 hours, more preferably between 6 and 24 hours.

### Multiple Compound Medicaments

An object of the present invention is to provide compounds capable of inducing hypothermia in an individual. The induction of hypothermia depends on the characteristics of the compounds and these characteristics may be to reach different target temperatures or different ranges of target temperatures, reaching the target temperatures and various speeds, the lifetime of the active compound and so on. It is therefore an object of the present invention to provide medicaments comprising more than one compound, such as at least two, at least three or at least four compounds as herein described.

The medicament may thus comprise compounds of the present invention wherein at least one compound induces hypothermia rapidly, or alternatively wherein at least one compound induces hypothermia slowly. In the present context rapidly means within few hours, such as within 2 hours, such as within 1 hours, whereas slowly means after several hours.

### Second Active Ingredient

An embodiment of the present invention is a pharmaceutical composition comprising a compound as herein described and furthermore comprising a second active ingredient. The second active ingredient may increase the hypothermic effect of the compound of the invention, or may have an alternative medical effect such as inducing pain relief or vasodilation.

The second active ingredient may thus be selected from the non-limiting group of: capsaicinoids, neurotensins, analgesics, opiods, GABAs and adrenergic antagonists.

Examples of these include, but are not limited to: Capsaicin (8-methyl-N-vanillyl-6-nonenamide) and neurotensin analogues KK13 and KK14.

### Kit of Parts

Another embodiment of the present invention comprises a kit of parts, wherein the kit includes at least one compound according to any of the above, a means for administering said compound and the instruction(s) on how to do so. It is within the scope of the present invention to include multiple dosages of the same composition or several different compositions. In a preferred embodiment the kit of parts further comprises a second active ingredient.

### EXAMPLES

### Example 1

### Cardiac Arrest

A 57-year-old woman is brought into hospital 21 minutes after having collapsed without warning. Staff at the emergency room is alerted in advance. The patient is evaluated in the emergency room where the physician in charge decides that the patient shall receive hypothermia therapy immediately to minimize the risk of damage to the brain and other tissues. An intravenous bolus injection of HU-210 (e.g. 100 microgram/kg body weight) or delta-8-THC phosphate (e.g. 40 mg/kg body weight) is administered.

The purpose of hypothermia therapy is to lower the patient's core body temperature to 32-34 degrees Celsius for 12 to 24 hours (current American Heart Association recommendation). Depending on the individual's response to the medication 1-4 additional intravenous bolus injections may be required (HU-210: Additional injections of 20-100 microgram/kg body weight; delta-B-THC phosphate: Additional injections of 8-40 mg/kg body weight). Additional bolus injection may be administered after 6-12 hours from the first bolus injection.

At the hospital, concurrent treatments and examinations are not influenced by the administration of the hypothermia-inducing drug. Such treatments and examinations proceed uninterrupted.

### Example 2

### Perinatal Asphyxia

A newborn baby suffers cerebral ischemia during delivery as the umbilical cord gets wrapped around his neck. The APGAR score 10 minutes after delivery is 6. The pediatrician decides that the patient shall receive hypothermia therapy immediately to minimize the risk of damage to the brain and other tissues. An intravenous bolus injection of HU-210 (e.g. 100 microgram/kg body weight) or delta-8-THC phosphate (e.g. 40 mg/kg body weight) is administered. Additional bolus injection may be administered after 6-12 hours from the first bolus injection.

The purpose of hypothermia therapy is to lower the patient's core body temperature to 32-34 degrees Celsius for 12 to 24 hours (current American Heart Association recommendation). Depending on the individual's response to the medication 1-4 additional intravenous bolus injections may be required (HU-210: Additional injections of 20-100 microgram/kg body weight; delta-8-THC phosphate: Additional injections of 8-40 mg/kg body weight). Additional bolus injection may be administered after 6-12 hours from the first bolus injection.

At the hospital, concurrent treatments and examinations are not influenced by the administration of the hypothermia-inducing drug. Such treatments and examinations proceed uninterrupted.

### Example 3

### Stroke

A 72-year-old is brought to hospital 1 hour and 30 minutes after waking up with the entire right side of his body feeling numb and weak. The patient is evaluated in the neurology department and the physician in charge decides, suspecting a stroke, that the patient shall receive hypothermia therapy immediately to lessen damage to the brain. An intravenous bolus injection of HU-210 (e.g. 100 microgram/kg body weight) or delta-8-THC phosphate (e.g. 40 mg/kg body weight) is administered.

The purpose of hypothermia therapy is to lower the patient's core body temperature to 32-34 degrees Celsius for 12 to 24 hours (current American Heart Association recommendation). Depending on the individual's response to the medication 1-4 additional intravenous bolus injections may be required (HU-210: Additional injections of 20-100 microgram/kg body weight; delta-8-THC phosphate: Additional injections of 8-40 mg/kg body weight). Additional bolus injection may be administered after 6-12 hours from the first bolus injection.

At the hospital, concurrent treatments and examinations are not influenced by the administration of the hypothermia-inducing drug. Such treatments and examinations proceed uninterrupted.

### Example 4

### Myocardial Infarction

A 48-year-old man is brought to hospital 35 minutes after experiencing sudden severe chest pain, shortness of breath, and very unpleasant palpitations. Staff at the emergency room is alerted in advance. The patient is evaluated and the cardiologist in charge decides that the patient shall receive hypothermia therapy immediately to lessen damage to the heart and other tissues. An intravenous bolus injection of HU-210 (e.g. 100 microgram/kg body weight) or delta-8-THC phosphate (e.g. 40 mg/kg body weight) is administered.

The purpose of hypothermia therapy is to lower the patient's core body temperature to 32-34 degrees Celsius for 12 to 24 hours (current American Heart Association recommendation). Depending on the individual's response to the medication 14 additional intravenous bolus injections may be required (HU-210: Additional injections of 20-100 microgram/kg body weight; delta-8-THC phosphate: Additional injections of 840 mg/kg body weight). Additional bolus injection may be administered after 6-12 hours from the first bolus injection.

At the hospital, concurrent treatments and examinations are not influenced by the administration of the hypothermia-inducing drug. Such treatments and examinations proceed uninterrupted.

### Example 5

### Porcine Study Model

In order to evaluate an effective hypothermic dose of a cannabinoid receptor agonist compound according to the invention, the compound may be tested in the porcine study model. The porcine model is used because the body weight of the pigs is comparable to the body weight of humans. The efficacy of a compound tested in the porcine model may be correlated with the efficacy of HU-210 or delta-8-THC phosphate tested in the same porcine study model.

The individual cannabinoids that may be examined may be selective CB1 agonists, mixed CB1 and CB2 agonists, or any other combination covered by this invention.

### Study Subjects

The evaluation is carried out on “dansk landrace” pigs with a body weight of 70-90 kilo. The pigs will not be sedated; they will be fed twice a day; and they will be subjected to a day cycle consisting of 12 hours of light followed by 12 hours of dark.

### Drug Administration

The cannabinoid investigated are administered i.v. as bolus injections and may consist of 1 solitary injection, alternatively 2-4 repeated injections within a timeframe of 24 hours from the initial injection.

Generally 4 different doses plus vehicle are tested producing varying degrees of hypothermic responses.

### Hypothermic Effect

The primary effect evaluated is hypothermia. Temperature is measured using a temperature probe that is surgically positioned in a femoral artery two weeks prior to -commencement of the study. The probe is connected to telemetry equipment (e.g. implanted telemetry from Data Sciences International) ensuring the required readouts.

Temperature is measured every 15 minutes from 1 hour prior to drug administration to 12 hours after drug administration, and every 30 minutes subsequently until 24 hours after drug administration. Temperature measurement will be conducted via a permanent femoral artery temperature probe (telemetry).

The minimum temperature as well as a graph of the temperature at each point of measurement is recorded for each dose of cannabinoid.

### Other Effects

Blood pressure, heart rate and ECG will be registered every 15 minutes from 1 hour prior to drug administration to 12 hours after drug administration, and every 30 minutes subsequently until 24 hours after drug administration.

### REFERENCES

[1] Busto R, Dietrich W D, Globus M Y, Valdes I, Scheinberg P, Ginsberg M D: Small differences in intraischemic brain temperature critically determine the extent of ischemic neuronal injury. J Cereb Blood Flow Metab 1987; 7(6):729-738.
[2] Barone F C, Feuerstein G Z, White R F: Brain cooling during transient focal ischemia provides complete neuroprotection. Neurosci Biobehav Rev 1997; 21(1):31-44.
[3] Onesti S T, Baker C J, Sun P P, Solomon R A: Transient hypothermia reduces focal ischemic brain injury in the rat. Neurosurgery 1991; 29(3):369-373.
[4] Coimbra C, Wieloch T: Moderate hypothermia mitigates neuronal damage in the rat brain when initiated several hours following transient cerebral ischemia. Acta Neuropathol (Berl) 1994; 87(4):325-331.
[5] Zhang Y, Wong K C, Zhang Z: The effect of intraischemic mild hypothermia on focal cerebral ischemia/reperfusion injury. Acta Anaesthesiol Sin 2001; 39(2):65-69.
[6] Yamashita K, Eguchi Y, Kajiwara K, Ito H: Mild hypothermia ameliorates ubiquitin synthesis and prevents delayed neuronal death in the gerbil hippocampus. Stroke 1991; 22(12):1574-1581.
[7] Ooboshi H, Ibayashi S, Takano K, Sadoshima S, Kondo A, Uchimura H, Fujishima M: Hypothermia inhibits ischemia-induced efflux of amino acids and neuronal damage in the hippocampus of aged rats. Brain Res 2000; 884(1--2):23-30.
[8] Colbourne F, Corbett D, Zhao Z, Yang J, Buchan AM: Prolonged but delayed postischemic hypothermia: a long-term outcome study in the rat middle cerebral artery occlusion model. J Cereb Blood Flow Metab 2000; 20(12):1702-1708.
[9] Kawai N, Okauchi M, Morisaki K, Nagao S: Effects of delayed intraischemic and postischemic hypothermia on a focal model of transient cerebral ischemia in rats. Stroke 2000; 31(8):1982-1989.
[10] Maier C M, Sun G H, Kunis D, Yenari M A, Steinberg G K: Delayed induction and long-term effects of mild hypothermia in a focal model of transient cerebral ischemia: neurological outcome and infarct size. J Neurosurg 2001; 94(1):90-96.
[11] Maier C M, Ahern K, Cheng M L, Lee J E, Yenari M A, Steinberg G K: Optimal depth and duration of mild hypothermia in a focal model of transient cerebral ischemia: effects on neurologic outcome, infarct size, apoptosis, and inflammation. Stroke 1998; 29(10):2171-2180.
[12] Mild therapeutic hypothermia to improve the neurologic outcome. after cardiac arrest: N EngI J Med 2002; 346(8):549-556.
[13] Bernard S A, Gray T W, Buist M D, Jones B M, Silvester W, Gutteridge G, Smith K: Treatment of comatose survivors of out-of-hospital cardiac arrest with induced hypothermia. N Engl J Med 2002; 346(8):557-563.
[14] Bernard S: Therapeutic hypothermia after cardiac arrest: now a standard of care. Crit Care Med 2006; 34(3):923-924.
[15] Dietrich W D, Halley M, Valdes I, Busto R: Interrelationships between increased vascular permeability and acute neuronal damage following temperature-controlled brain ischemia in rats. Acta Neuropathol (Berl) 1991; 81(6):615-625.
[16] Karibe H, Zarow G J, Graham S H, Weinstein P R: Mild intraischemic hypothermia reduces postischemic hyperperfusion, delayed postischemic hypoperfusion, blood-brain barrier disruption, brain edema, and neuronal damage volume after temporary focal cerebral ischemia in rats. J Cereb Blood Flow Metab 1994; 14(4):620-627.
[17] Huang Z G, Xue D, Preston E, Karbalai H, Buchan A M: Biphasic opening of the blood-brain barrier following transient focal ischemia: effects of hypothermia. Can J Neurol Sci 1999; 26(4):298-304.
[18] Bagenholm R, Nilsson U A, Kjellmer I: Formation of free radicals in hypoxic ischemic brain damage in the neonatal rat, assessed by an endogenous spin trap and lipid peroxidation. Brain Res 1997; 773(1-2):132-138.
[19] Kil H Y, Zhang J, Piantadosi C A: Brain temperature alters hydroxyl radical production during cerebral ischemia/reperfusion in rats. J Cereb Blood Flow Metab 1996; 16(1):100-106.
[20] Kumura E, Yoshimine T, Takaoka M, Hayakawa T, Shiga T, Kosaka H: Hypothermia suppresses nitric oxide elevation during reperfusion after focal cerebral ischemia in rats. Neurosci Lett 1996; 220(1):45048.
[21] Baker A J, Zornow M H, Grafe M R, Scheller M S, Skilling S R, Smullin D H, Larson A A: Hypothermia prevents ischemia-induced increases in hippocampal glycine concentrations in rabbits. Stroke 1991; 22(5):666-673.
[22] Takagi K, Ginsberg M D, Globus M Y, Dietrich W D, Martinez E, Kraydieh S, Busto R: Changes in amino acid neurotransmitters and cerebral blood flow in the ischemic penumbral region following middle cerebral artery occlusion in the rat: correlation with histopathology. J Cereb Blood Flow Metab 1993; 13(4):575-585.
[23] Nakashima K, Todd M M: Effects of hypothermia on the rate of excitatory amino acid release after ischemic depolarization. Stroke 1996; 27(5):913-918.
[24] Maier C M, Sun G H, Cheng D, Yenari M A, Chan P H, Steinberg G K: Effects of mild hypothermia on superoxide anion production, superoxide dismutase expression, and activity following transient focal cerebral ischemia. Neurobiol Dis 2002; 11(1):28-42.
[25] Pulsinelli W: Pathophysiology of acute ischaemic stroke. Lancet 1992; 339(8792):533-536.
[26] Eguchi Y, Yamashita K, Iwamoto T, Ito H: Effects of brain temperature on calmodulin and microtubule-associated protein 2 immunoreactivity in the gerbil hippocampus following transient forebrain ischemia. J. Neurotrauma 1997; 14(2):109-118.
[27] Sick T J, Tang R, Perez-Pinzon M A: Cerebral blood flow does not mediate the effect of brain temperature on recovery of extracellular potassium ion activity after transient focal ischemia in the rat. Brain Res 1999; 821 (2):400-406.
[28] Hu B R, Kamme F, Wieloch T: Alterations of Ca2+/calmodulin-dependent protein kinase II and its messenger RNA in the rat hippocampus following normo- and hypothermic ischemia. Neuroscience 1995; 68(4):1003-1016.
[29] Busto R, Globus M Y, Neary J T, Ginsberg M D: Regional alterations of protein kinase C activity following transient cerebral ischemia: effects of intraischemic brain temperature modulation. J Neurochem 1994; 63(3):1095-1103.
[30] Ishikawa M, Sekizuka E, Sato S, Yamaguchi N, Inamasu J, Bertalanffy H, Kawase T, Iadecola C: Effects of moderate hypothermia on leukocyte- endothelium interaction in the rat pial microvasculature after transient middle cerebral artery occlusion. Stroke 1999; 30(8):1679-1686.
[31] Inamasu J, Suga S, Sato S, Horiguchi T, Akaji K, Mayanagi K, Kawase T: Post-ischemic hypothermia delayed neutrophil accumulation and microglial activation following transient focal ischemia in rats. J Neuroimmunol 2000; 109(2):66-74.
[32] Hara A, Niwa M, lwai T, Yano H, Bunai Y, Uematsu T, Yoshimi N, Mori H: Increase of fragmented DNA transport in apical dendrites of gerbil CA1 pyramidal neurons following transient forebrain ischemia by mild hypothermia. Neurosci Lett 2000; 280(1):73-77.
[33] Xu L, Yenari M A, Steinberg G K, Giffard R G: Mild hypothermia reduces apoptosis of mouse neurons in vitro early in the cascade. J Cereb Blood Flow Metab 2002; 22(1):21-28.
[34] Zhang Z, Sobel R A, Cheng D, Steinberg G K, Yenari M A: Mild hypothermia increases Bcl-2 protein expression following global cerebral ischemia. Brain Res Mol Brain Res 2001; 95(1-2):75-85.
[35] Niwa M, Hara A, Iwai T, Nakashima M, Yano H, Yoshimi N, Mori H, Uematsu T: Relationship between magnitude of hypothermia during ischemia and preventive effect against post-ischemic DNA fragmentation in the gerbil hippocampus. Brain Res 1998; 794(2):338-342.
[36] Inamasu J, Suga S, Sato S, Horiguchi T, Akaji K, Mayanagi K, Kawase T: Postischemic hypothermia attenuates apoptotic cell death in transient focal ischemia in rats. Acta Neurochir Suppl 2000; 76:525-527.
[37] Leker R. R, Gai N, Mechoulam R, Ovadia H: Drug-induced hypothermia reduces ischemic damage: effects of the cannabinoid HU-210. Stroke 2003; 34(8):2000-2006.
[38] Ovadia H, Wohlman A, Mechoulam R, Weidenfeld J: Characterization of the hypothermic effect of the synthetic cannabinoid HU-210 in the rat. Relation to the adrenergic system and endogenous pyrogens. Neuropharmacology 1995; 34(2):175-180.
[39] Maas A I, Murray G, Henney H, III, Kassem N, Legrand V, Mangelus M, Muizelaar JP, Stocchetti N, Knoller N: Efficacy and safety of dexanabinol in severe traumatic brain injury: results of a phase III randomised, placebo-controlled, clinical trial. Lancet Neurol 2006; 5(1):38-45.
[40] Huestis M A, Gorelick D A, Heishman S J, Preston K L, Nelson R A, Moolchan E T, Frank R A: Blockade of effects of smoked marijuana by the CB1-selective cannabinoid receptor antagonist SR141716. Arch Gen Psychiatry 2001; 58(4):322-328.
[41] Hosko M J, Schmeling W T, Hardman H F: Evidence for a Caudal Brainstem Site of Action for Cannabinoid Induced Hypothermia. Brain Research Bulletin. 1981, Vol. 6, p. 253.
[42] Howlett A C, Barth, F, Bonner, T I, Cabral, G, Casellas, P, Devane; W A, Felder; C C, Herkenham, M, Mackie, K, Martin, B R, Mechoulam, R, and Pertwee, E G: International Union of Pharmacology, XXVII. Classification of Cannabinoid Receptors, Pharmacological Reviews, 2002, Vol. 54, No. 2, 161-202.
[43] Herkenham, M, Lynn, A B, Johnson, M R, Melvin, L S, de Costa, B R, and Rice, K C: Cannabinoid Receptor Localization in Brain, PNAS 1990; 87; 1932-1936.

1. A cannabinoid receptor agonist compound for use in induction of hypothermia in a human being.

2. The cannabinoid receptor agonist compound according to claim 1, wherein the compound is a cannabinoid of the general formula: 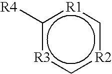
wherein R1, R2, R3 and R4 individually is a chemical moiety or a chemical bond.

3. The cannabinoid receptor agonist compound as defined in claim 2, wherein R1 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl or phosphate, optionally further substituted one or more times with C, S, N, O, OH, phenyl, amine (NH), halogen, methyl, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, or nitro, and preferably is C, O, N optionally substituted with O, OH, alkyl, alkenyl, alkynyl, or phosphate, optionally further substituted with methyl, alkyl or phosphate and more preferably is C, optionally substituted with H, OH, OCH3 or phosphate.

4. The cannabinoid receptor agonist compound as defined in claim 2, wherein R2 is selected from the group of: C, S, N, O, optionally substituted with C, S, N, O, P, OH, hydrogen, alkyl, alkenyl, alkynyl, any of which may or may not be branched or comprise substituents such as phosphate, cycloalkyl, heterocycloalkyl, cycloalkenyl, methyl, dimethyl, or may be further substituted one or more times with C, S, N, O, P, OH, hydrogen, alkyl, alkenyl, alkynyl, sulfonyl, any of which may or may not be branched or comprise substituents such as hydrogen, alkyl, alkenyl, alkynyl, fluoride, phosphate, cycloalkyl, heterocycloalkyl, cycloalkenyl, dimethyl, phenyl and preferably is C substituted with C, O, P, H, OH, OSO2, phosphate, alkyl, alkenyl, alkynyl such as (C1-CX), phenyl any of which may be substituted with methyl, dimethyl, sulfonyl, cycloalkyl, hetereocycloalkyl, cycloalkenyl, fluoride, phenyl, phosphate, and more preferably is C substituted with C, O, OSO2, alkyl such as (C3-C11) any of which may be further substituted with methyl, dimethyl, alkyl such as (C1-CX), phenyl, phosphate or further substituted by fluoride, phosphate, methyl, dimethyl and wherein X is an integer of from 1 to 20.

5. The cannabinoid receptor agonist compound as defined in claim 2, wherein R3 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkenyl, phosphate, optionally further substituted one or more times with C, S, N, O, OH, methyl, phenyl, diheterocycle, amine (NH), halogen, substituted lower alkyl, aryl, lower alcohol, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, or nitro, preferably is C, O, N, S, optionally substituted with O, OH, alkyl, alkenyl, alkynyl, or phosphate, optionally further substituted one or more times with methyl, diheterocycle, lower alcohol, alkyl or phosphate more preferably is C which may be substituted with C, O, N, OH, phosphate, any of which may be substituted one or more times with C, ethyl, methyl, phosphate, diheterocycle, lower alcohol, alkyl such as (C1-C2) wherein C2 binds to R4 when R4 is C, optionally further substituted by methyl, dimethyl or phosphate.

6. The cannabinoid receptor agonist compound as defined in claim 2, wherein R4 is selected from the group of: C, H, S, N, O, optionally substituted with C, H, S, N, O, P, OH, hydrogen, alkyl, alkenyl, alkynyl, phenyl, diphenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, phosphate, optionally further substituted one or more times with C, S, N, O, OH, phenyl, amine (NH), halogen, substituted lower alkyl, alkyl such as (C1-CX) any of which may be further substituted with methyl, dimethyl, alkyl such as (C1-CX), phenyl, phosphate or further substituted by fluoride, phosphate, methyl, dimethyl, aryl, heterocyclyl, heteroaryl, aryl-(C1-4)-alkyl, heteroaryl-(C1-4)-alkyl, heterocyclyl-(C1-4)-alkyl, cycloalkylalkyl, dicycloalkyl, tricycloalkyl, cycloalkenyl, alkoxy, carboxy, halogen, trifluoromethyl, cyano, amino, or nitro, (alcohol) and preferably is C, H, N, O optionally substituted with alkyl, alkenyl, alkynyl, alcohol, phenyl, diphenyl, dicycloalkyl, tricycloalkyl, cycloalkenyl any of which may bond with R1 or R3 forming a ring, optionally further substituted with one or more alkyl, alkenyl, alkynyl, OH, and more preferably is C, H, (C1-Cy), dicycloalkyl, or tricycloalkyl, cycloalkenyl any C of which may bond with R1 or R3 forming a ring, and optionally is substituted with methyl, dimethyl, phenyl, diphenyl, optionally further substituted with alkyl and/or OH and wherein X is an integer of from 1 to 15 and y is an integer of from 1 to 8.

7. The cannabinoid receptor agonist compound as defined in claim 2, wherein R1 is C, O, N optionally substituted with O, OH, alkyl, alkenyl, alkynyl, or phosphate, optionally further substituted with methyl, alkyl or phosphate.

8. The cannabinoid receptor agonist compound as defined in claim 2, wherein R1 is C, optionally substituted with H, OH, OCH3 or phosphate, when R2 as defined in is C substituted with C, O, OSO2, alkyl such as (C3-C11) any of which may be further substituted with methyl, dimethyl, alkyl such as (C1-CX), phenyl, phosphate or further substituted by fluoride, phosphate, methyl, dimethyl when R3 is C which may be substituted with C, O, N, OH, phosphate, any of which may be substituted with C, ethyl, phosphate, alkyl such as (C1-C2) wherein C2 binds to R4 when R4 is C, optionally further substituted by methyl, dimethyl or phosphate.

9. The cannabinoid receptor agonist compound according to claim 1, wherein the compound is a cannabinoid of the general formula: 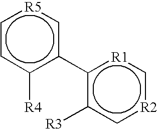
wherein R1, R2, R3, R4 and R5 individually is a chemical moiety or a chemical bond.

10. The cannabinoid receptor agonist compound as defined in claim 9, wherein R1 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, phosphate, optionally bonding with the C in the ring next to R5, optionally further substituted one or more times with C, S, N, O, OH, phenyl, phosphate, amine (NH), halogen, methyl, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, or nitro, and preferably is C, O, N optionally substituted with O, OH, alkyl, alkenyl, alkynyl, or phosphate, optionally further substituted with alkyl or phosphate and more preferably is C, optionally substituted one or more times with H, O, OH, OCH3 or phosphate.

11. The cannabinoid receptor agonist compound as defined in claim 9, wherein R2 is selected from the group of: C, S, N, O, optionally substituted with C, S, N, O, P, OH, hydrogen, alkyl, alkenyl, alkynyl, any of which may or may not be branched or comprise substituents such as phosphate, heterocycloalkyl, cycloalkyl, cycloalkenyl, methyl, dimethyl, or may be further substituted with C, S, N, O, P, OH, hydrogen, alkyl, alkenyl, alkynyl, sulfonyl, any of which may or may not be branched or comprise substituents such as hydrogen, alkyl, alkenyl, alkynyl, fluoride, phosphate, heterocycloalkyl, cycloalkyl, cycloalkenyl, dimethyl, phenyl and preferably is C substituted with C, O, P, H, OH, OSO2, phosphate, alkyl, alkenyl, alkynyl such as (C1 -CX), phenyl any of which may be substituted with methyl, dimethyl, sulfonyl, hetereocycloalkyl, fluoride, phenyl, phosphate, and more preferably is C substituted with C, O, OSO2, alkyl such as (C3-C11) any of which may be further substituted with methyl, dimethyl, alkyl such as (C1-CX), phenyl, phosphate or further substituted by fluoride, phosphate, methyl, dimethyl and wherein X is an integer of from 1 to 15.

12. The cannabinoid receptor agonist compound as defined in claim 9, wherein R3 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, or nitro, any of which may connect with R4 and preferably is C, O, N, OH, phosphate optionally substituted one or more times with alkyl, OH, phosphate any of which may connect with R4 and more preferably is O, OH, NH, optionally connecting with R4 thus forming a ring.

13. The cannabinoid receptor agonist compound as defined in claim 9, wherein R4 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, or nitro, any of which may connect with R3 and preferably is C, N, O, P, OH, lower substituted alkyl, alkenyl, alkynyl, phenyl, optionally substituted with OH, methyl, dimethyl any of which may connect with R3 and more preferably is C, optionally connecting with R3 and optionally substituted with methyl, dimethyl or methyn.

14. The cannabinoid receptor agonist compound as defined in claim 9, wherein R5 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, phosphate, optionally bonding with R1, optionally further substituted one or more times with C, S, N, O, OH, phenyl, amine (NH), halogen, methyl, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, phosphate or nitro, and preferably is C, N, O, optionally substituted with C, O, CH2OH, methyl, dimethyl, alkyl, alkenyl, alkynyl, phenyl, phosphate and more preferably is C, CO, optionally substituted with C, methyl, methyn (CH2), optionally substituted with CH2OH.

15. The cannabinoid receptor agonist compound as defined in claim 9, wherein R1 is C, O, N optionally substituted with O, OH, alkyl, alkenyl, alkynyl, or phosphate, optionally further substituted with alkyl or phosphate, when R2 is C substituted with C, O, P, H, OH, OSO2, phosphate, alkyl, alkenyl, alkynyl such as (C1-CX), phenyl any of which may be substituted with methyl, dimethyl, sulfonyl, hetereocycloalkyl, fluoride, phenyl or phosphate, when R3 is C, O, N, OH, phosphate optionally substituted one or more times with alkyl, OH, phosphate any of which may connect with R4 thus forming a ring.

16. The cannabinoid receptor agonist compound according to claim 9, wherein R1 preferably is C, optionally substituted with H, OH, OCH3 or phosphate when R2 is C substituted with C, O, OSO2, alkyl such as (C3-C8) any of which may be further substituted with methyl, dimethyl, alkyl such as (C1-CX), phenyl, phosphate or further substituted by fluoride, phosphate, methyl, dimethyl when R3 is O, OH, NH, optionally connecting with R4, when R4 is C, optionally connecting with R3 and optionally substituted with methyl, dimethyl or methyn, when R5 preferably is C, CO, optionally substituted with C, methyl, methyn (CH2), optionally substituted with CH2OH and wherein X is an integer of from 1 to 15.

17. The cannabinoid receptor agonist compound as defined in claim 1, wherein the compound has the general formula:
R1—R2
Wherein, R1 is (C1-Cx) saturated or unsaturated, and optionally is substituted one or more times with lower alkyl, alkenyl, alkynyl, O, OH, N, when R2 is C, N, O, NH2 optionally substituted one ore more times with lower alkyl, alkenyl, alkynyl, phenyl, OH, NH2 cycloalkane, methyl or OCH3 and wherein x is an integer of from 1 to 30.

18. The cannabinoid receptor agonist compound as defined in claim 1, wherein the compound has the general formula:
R1—R2
wherein R1 preferably is (C1-CX), is saturated or unsaturated and optionally substituted with methyl, dimethyl, O, or N when R2 is N, O, NH2 optionally substituted with C, CH2OH, CH(CH2)2 (cyclopropane), optionally further substituted one or more times with CH2OH, CH2Cl and wherein X is an integer of from 1 to 21.

19. The cannabinoid receptor agonist compound according to claim 1, wherein the compound is an aminoalkylindole of the general formula: 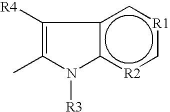
wherein R1, R2, R3 and R4 individually is a chemical moiety or a chemical bond.

20. The cannabinoid receptor agonist compound as defined in claim 19, wherein R1 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, or nitro, and preferably is C, O, N optionally substituted with O, phosphate, N, C, lower alkyl, OH, optionally further substituted with lower alkyl, OH, phosphate and more preferably is C, substituted with O, further substituted with methyl.

21. The cannabinoid receptor agonist compound as defined in claim 19, wherein R2 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, or nitro, any of which may bond with R3, and preferably is C, N, O, optionally substituted with C, O, N, phosphate, lower alkyl optionally further substituted with lower alkyl, OH, phosphate, any of which may bond with R3 and more preferably is C, substituted with O, further substituted with C optionally bond forming with R3.

22. The cannabinoid receptor agonist compound as defined in claim 19, wherein R3 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, or nitro, any of which may bond R2 and preferably is C, N, O, alkyl, alkenyl, alkynyl, optionally substituted with C, N, O, OH, phosphate, halogen any of which may bond R2 and more preferably is (C1-Cx) and wherein x is an integer of from 1 to 3, optionally substituted one or more times with O, dichloro-phenyl or morpholine and any of which may bond R2.

23. The cannabinoid receptor agonist compound as defined in claim 19, wherein R4 is selected from the group of: C, S, N, O, optionally substituted with C, S, N, O, P, OH, hydrogen, alkyl, alkenyl, alkynyl, phenyl, benzyl, amine (NH), halogen, substituted lower alkyl, aryl, heterocycloalkyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, phosphate, optionally further substituted one or more times with C, S, N, O, OH, phenyl, amine (NH), halogen, methyl, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, cycloalkenyl, alkoxy, carboxy, halogen, trifluoromethyl, cyano, amino, or nitro, and preferably is C, N, O optionally substituted with C, N, O, OH, lower alkyl, alkenyl, alkynyl, phosphate, optionally further substituted one or more times with O, OH, phenyl, diphenyl, morpholino, and halogen, and more preferably is C, optionally substituted with C, O and/or diphenyl, optionally further substituted with morpholine.

24. The cannabinoid receptor agonist compound as defined in claim 19, wherein R1 is C, O, N optionally substituted with O, phosphate, N, C, lower alkyl, OH, optionally further substituted with lower alkyl, OH or phosphate, when R2 is C, N, O, optionally substituted with C, O, N, phosphate, lower alkyl optionally further substituted with lower alkyl, OH, phosphate, any of which may bond with R3, when R3 is C, N, O, alkyl, alkenyl, alkynyl, optionally substituted with C, N, O, OH, phosphate, halogen any of which may bond R2, when R4 is C, N, O optionally substituted with C, N, O, OH, lower alkyl, alkenyl, alkynyl, phosphate, optionally further substituted one or more times with O, OH, phenyl, diphenyl, morpholino, and/or halogen.

25. The cannabinoid receptor agonist compound as defined in claim 19, wherein R1 preferably is C, substituted with O, further substituted with methyl when R2 is C, substituted with O, further substituted with C optionally bond forming with R3 when R3 is (C1-Cx) and wherein x is an integer of from 1 to 3, optionally substituted one or more times with O, dichloro-phenyl or morpholine when R4 is C, optionally substituted with C, O and/or diphenyl, optionally further substituted with morpholine.

26. The cannabinoid receptor agonist compound according to claim 1, wherein the cannabinoid receptor is CB1 and/or CB2.

27. The cannabinoid receptor agonist compound according to claim 1, wherein the cannabinoid receptor is CB1.

28. The cannabinoid receptor agonist compound according to claim 1, which is hydrophilic.

29. The cannabinoid receptor agonist compound according to claim 1, wherein the medicament induces hypothermia of between 32 and 36 degree Celsius.

30. The cannabinoid receptor agonist compound according to claim 1, wherein the treatment comprises administration of at least two compounds.

31. The cannabinoid receptor agonist compound according to claim 30, wherein at least one compound induces hypothermia rapidly.

32. The cannabinoid receptor agonist compound according to claim 31, wherein at least one compound induces hypothermia slowly.

33. The cannabinoid receptor agonist compound according to claim 1, wherein the treatment comprises administration of a second active ingredient.

34. The cannabinoid receptor agonist compound according to claim 33, wherein the second active ingredient is selected from the group of: capsaicinoids, neurotensins, analgesics, opiods, GABAs and adrenergic antagonists.

35. The cannabinoid receptor agonist compound according to claim 1, for administration by injection, suppository, oral administration, sublingual tablet or spray, cutaneous administration, or inhalation.

36. The cannabinoid receptor agonist compound according to claim 35, wherein the injection is intravenous, intramuscular, intraspinal, intraperitoneal, subcutaneous, a bolus or a continuous administration.

37. The cannabinoid receptor agonist compound according to claim 1, wherein administration occurs at intervals of 30 minutes to 24 hours.

38. The cannabinoid receptor agonist compound according to claim 1, wherein administration occurs at intervals of 1 to 6 hours.

39. The cannabinoid receptor agonist compound according to claim 1, wherein the duration of the treatment is from 6 to 72 hours.

40. The cannabinoid receptor agonist compound according to claim 1, wherein the dosage of the medicament is between 10 μg to 10 mg pr kg body mass.

41. Cannabinoid receptor agonist compound for use in induction of hypothermia in a human being for treatment of ischemia, wherein the cannabinoid receptor agonist compound is as defined in claim 1.

42. Cannabinoid receptor agonist compound for use in induction of hypothermia in a human being for suffering from or at risk of suffering from ischemia, wherein the cannabinoid receptor agonist compound is as defined in claim 1.

43. The cannabinoid receptor agonist compound according to claim 41, for prophylaxis and/or treatment of ischemia in connection with cardiovascular diseases, asphyxia and/or traumatic brain injuries.

44. The cannabinoid receptor agonist compound according to claim 43, wherein the ischemia is due to cardiovascular diseases such as: myocardial infarction, acute coronary syndrome, cardiac arrest, stroke, arterial aneurism, subarachnoid haemorrhage, arteriosclerosis, angina pectoris, hypertension, hypercholesterolemia, cardiac arrhythmia, cardiomegaly, cardiomyopathy, heart valve regurgitation and heart valve stenosis.

45. The cannabinoid receptor agonist compound according to claim 43, wherein the ischemia is due to asphyxia such as: perinatal asphyxia and/or non-perinatal asphyxia.

46. A pharmaceutical composition comprising at least one cannabinoid receptor agonist compound according to claim 1, and optionally a pharmaceutically acceptable carrier.

47. The pharmaceutical composition according to claim 46, comprising a second active ingredient.

48. The pharmaceutical composition according to claim 47, wherein the second active ingredient is selected from the group of: capsaicinoids, neurotensins, analgesics, opiods, GABAs and adrenergic antagonists.

49. The pharmaceutical composition according to claim 46, wherein the pH of the composition is between pH 5 and pH 9.

50. The pharmaceutical composition according to claim 46, formulated for administration by injection, suppository, oral administration, sublingual tablet or spray, cutaneous administration, or inhalation.

51. The pharmaceutical composition according to claim 46, formulated for injection, wherein the injection is intravenous, intramuscular, intraspinal, intraperitoneal, subcutaneous, a bolus or a continuous administration.

52. A kit of parts comprising at least two cannabinoid receptor agonist compounds as defined in claim 1.

53. A kit of parts comprising at least one cannabinoid receptor agonist compound as defined in claim 1, and a second active ingredient.

54. The kit of parts according to claim 53, wherein the second active ingredient is selected from the group of: capsaicinoids, neurotensins, analgesics, opiods, GABAs and adrenergic antagonists.

55. A cannabinoid receptor antagonist for use in reverting hypothermia in a human being.
